(19) United States
(12) Patent Application Publication
(10) Pub. No.: US 2025/0049975 A1
(43) Pub. Date: Feb. 13, 2025

(54) STEAM GENERATION IN A SYSTEM FOR TREATING CONTAINERS

(71) Applicant: KRONES AG, Neutraubling (DE)

(72) Inventors: Juergen Soellner, Neutraubling (DE); Reiner Gloetzl, Neutraubling (DE); Bernd Schafaczek, Neutraubling (DE)

(21) Appl. No.: 18/797,417

(22) Filed: Aug. 7, 2024

(30) Foreign Application Priority Data
Aug. 8, 2023 (DE) 10 2023 121 164.1

(51) Int. Cl. A61L 2/20; A61L 2/26; B01B 1/00

(52) U.S. Cl. CPC A61L 2/208; A61L 2/26; B01B 1/005; A61L 2202/11; A61L 2202/17; A61L 2202/23

(57) ABSTRACT

The disclosure relates to a system (1) for treating containers, preferably a beverage filling system, with a container treatment portion (50) which is configured to treat the containers, preferably to fill the containers with a filling product and/or to close the containers with a container closure, and in which condensate (K) accumulates during sterilization; a high-speed steam generator (10) with a steam boiler (15) which is configured to heat feed water, preferably by means of at least one electrical heating element (15a), and to vaporize it, wherein the high-speed steam generator (10) is configured to receive the condensate (K) accumulating in the container treatment portion (50) via a condensate line (11) and to introduce it into the steam boiler (15) as feed water to be vaporized during regular operation.

### CROSS-REFERENCE TO RELATED APPLICATIONS

This application claims priority from German Patent Application No. DE 10 2023 121 164.1, filed Aug. 8, 2023 in the German Patent and Trademark Office, the disclosure of which is incorporated herein by reference in its entirety.

### TECHNICAL FIELD

The present disclosure relates to a system for treating containers, preferably a beverage filling system, and to a method for providing steam in such a system.

### PRIOR ART

Vaporizers of various types of construction are known for evaporating liquid media; they are used, for example, in beverage filling systems to provide gaseous hydrogen peroxide for disinfecting system components that come into contact with the product. The vaporizers usually have a heated vaporizer surface, onto which is sprayed the medium, which is still liquid and then vaporizes. The vaporizer surface is heated by means of hot steam or electric heating elements to provide the desired temperature of the vaporizer surface.

A device for evaporating a fluid medium in a filling product filling system is disclosed, for example, in WO 2018/060422 A2.

The required steam pressure is provided by a steam boiler, which feeds the produced steam into a steam network. The pressure in the steam network is built for the highest steam pressure required by the connected consumers. For all other consumers, the steam pressure is reduced accordingly.

If a higher pressure than the pressure present in the steam network is only required in small quantities, a) either the pressure in the entire steam network can be increased, provided that the steam boiler is configured for the required pressure, or b) a separate, for example electrically operated, high-speed steam generator can be used for this amount of steam.

If the steam pressure is increased throughout the steam network in accordance with option a), it may be necessary to re-engineer and, if necessary, replace existing safety devices. Furthermore, operating the entire steam network at a high pressure, which is only required for a small amount at one consumer, does not make sense from an energy efficiency point of view. The steam pressure must be reduced at the other consumers, which entails losses.

If an additional (electric) high-speed steam generator is used in accordance with option b), the existing steam network can be operated at a lower pressure level, since the small amount of steam with a higher steam pressure is generated via a separate steam generator. To feed the steam generators, water from a reverse osmosis system, for example, is required to allow economical running time without a blow-down process. If the feed water quality is poor and contains salt, the steam generator must be frequently blown down. For this purpose, it is necessary to reduce the steam pressure, in particular for small buffer volumes, which leads to a short-term standstill of the filling system and a corresponding loss of system efficiency.

### Illustration of the Invention

An object of the invention is to propose an improved system for treating containers and an improved method for providing steam in such a system, in particular to improve the process of steam generation in terms of energy efficiency and accelerate it.

The object is achieved by a system having the features of claim 1 and a method having the features of the independent method claim. Advantageous further embodiments follow from the dependent claims, the following description of the invention and the description of various embodiments.

The invention relates to a system for treating containers with a container treatment portion which is configured to treat the containers, and a high-speed steam generator with a steam boiler which is configured to heat feed water, preferably by means of at least one electrical heating element, and to vaporize it.

The system is particularly preferably part of a beverage filling system, the container treatment portion in this case comprising in particular a filling device, for example for filling water (still or carbonated), beer, juice, soft drinks, smoothies, dairy products, mixed drinks and the like. The container treatment portion can comprise a filling device, a closing device, a stretch blow molding device for producing containers, a container cleaning system, a labeler, etc. or a combination of different devices, in particular a filling device for filling containers with a filling product and closing the containers with a container closure. The containers may be bottles made of glass, PET, cans, cartons or suitable containers of other types that can be filled with a liquid filling product, in particular a beverage.

In such a container treatment portion, condensate can accumulate during various processes, for example during a CIP (“Cleaning in Place”) treatment or an SIP (“Sterilization in Place”) treatment, in which the container treatment portion is cleaned and/or sterilized and/or disinfected without significant disassembly of the product-contact surfaces. In order to achieve sterilization, the media, such as compressed air, are heated via heat exchangers supplied with hot steam. Water condenses on the surfaces of the heat exchangers in the container treatment portion as soon as the steam is cooled below the dew point. In this way, condensate accumulates in the treatment portion during sterilization.

The high-speed steam generator according to the invention is now configured to receive the condensate accumulating in the container treatment portion during sterilization via a condensate line and to introduce it into the steam boiler as feed water to be vaporized.

The high-speed steam generator is thus operated at least partially, preferably exclusively, with condensate. This means that no separate water treatment is required or that any water treatment can be carried out with less effort, since the condensate already has a very low mineral content. Furthermore, the condensate already has a high temperature, for example approx. 90° C., which saves energy and accelerates steam generation compared to cold feed water.

The high-speed steam generator preferably has a feed vessel which is configured to receive the condensate via the condensate line, to collect it and to deliver it to the steam boiler. The condensate in the feed vessel can be fed to the steam boiler as required or continuously. For this purpose, a pressure booster pump is preferably provided, which is configured to pump the condensate from the feed vessel into the steam boiler.

The condensate collected in the feed vessel can preferably be drained via a drain pipe if necessary.

The steam produced in the high-speed steam generator can be used in various processes, in particular for operating a vaporizer which is configured, for example, to produce gaseous hydrogen peroxide for disinfecting containers to be filled or container closures.

The system preferably comprises a vaporizer which is configured to receive steam from the steam boiler of the high-speed steam generator and to vaporize a treatment agent, preferably hydrogen peroxide. In this way, the high-speed steam generator can be integrated into an existing system with little mechanical engineering effort.

The system comprises an operating steam generator which is different from the high-speed steam generator and is configured to generate operating steam. In particular, the container treatment portion can be supplied with operating steam from the operating steam generator, for example for sterilization during CIP and/or SIP treatment, in which the container treatment portion is cleaned and/or sterilized and/or disinfected without significant disassembly on the product-contact surfaces. The operating steam can perform alternative or additional functions in the container treatment portion, for example to heat certain parts of the system.

The temperature, for example approx. 160° C., and/or the pressure, for example approx. 6 bar, of the steam produced by the operating steam generator are preferably lower than the temperature, for example approx. 180° C., and/or the pressure, for example approx. 9 bar, of the steam produced by the high-speed steam generator. In this case, the high-speed steam generator can act as a “booster” for the operating steam generator, which means that certain processes, such as sterilization of container closures, can be carried out in a particularly energy-efficient and resource-efficient manner, since the entire steam network does not have to be supplied with steam at a higher pressure and temperature.

The high-speed steam generator preferably has a lower capacity than the operating steam generator. For example, the high-speed steam generator is configured to produce only a fraction of the steam of the operating steam generator, for example one hundredth of the amount of the operating steam generator.

Preferably, the operating steam generator is part of a cleaning circuit which is configured to clean and/or sterilize and/or disinfect the container treatment portion as part of a CIP treatment. The condensate used in the high-speed steam generator during regular operation is preferably generated during CIP and/or SIP treatment during sterilization.

Preferably, the high-speed steam generator is connected to the cleaning circuit of the system via a CIP feed line. Connection to an existing cleaning cycle, preferably an acid cleaning cycle, leads to longer service life without the need for a corresponding blow-down process. Any mineral residues that may accumulate on the heating elements can be automatically cleaned off during the next cleaning cycle of the container treatment portion.

Preferably, the connection to the cleaning circuit is made via a block & bleed valve combination to prevent the media from mixing in the event of a valve leak.

The above-mentioned object is further achieved by a method for providing steam in a system for treating containers, preferably in a beverage filling system. The method comprises: generating condensate in a container treatment portion during sterilization, wherein the container treatment portion is configured to treat the containers, preferably to fill the containers with a filling product and/or to close the containers with a container closure; during regular operation, introducing the condensate via a condensate line into a steam boiler of a high-speed steam generator; heating and evaporating the condensate as feed water in the steam boiler, preferably by means of at least one electrical heating element.

The features, technical effects, advantages and embodiments that have been described in relation to the system apply analogously to the method.

For the reasons mentioned above, the high-speed steam generator preferably has a feed vessel which receives the condensate via the condensate line, collects it and delivers it to the steam boiler. The condensate is preferably pumped from the feed vessel into the steam boiler by means of a booster pump.

For the reasons stated above, the steam generated in the steam boiler preferably drives a vaporizer which evaporates a treatment agent, preferably hydrogen peroxide, wherein the vaporizer preferably provides the vaporized treatment agent for sterilizing containers to be filled and container closures during regular operation.

Preferably, for the reasons stated above, an operating steam generator which is different from the high-speed steam generator produces operating steam whose temperature and/or pressure are preferably lower than the temperature and/or pressure of the steam produced by the high-speed steam generator.

Preferably, the operating steam generator is part of a cleaning circuit which cleans and/or sterilizes and/or disinfects the container treatment portion as part of a CIP treatment.

Preferably, the condensate accumulates during CIP treatment in the container treatment portion, wherein the condensate is collected in the feed vessel of the high-speed steam generator until later use. The condensate is thus collected during system cleaning and used at a later time. For example, several hours may elapse between the collection of condensate and the use of the condensate.

A distinction is made here between regular operation, i.e., the actual production in the system in which beverages are filled into containers, and a preparatory cleaning operation in which the system, in particular the container treatment portion, is subjected to CIP and/or SIP treatment for sterilization.

The condensate accumulates during the cleaning operation of the container treatment portion and is then collected in the feed tank. On this basis, the condensate collected in the feed vessel is preferably used during the regular operation of the container treatment portion. More specifically, the condensate collected in the feed vessel is preferably fed to the high-speed steam generator during regular operation of the container treatment portion, wherein the steam generated in the high-speed steam generator is then used to vaporize the treatment agent, preferably hydrogen peroxide, and the vaporized treatment agent is fed to the container treatment portion during regular operation.

The condensate which accumulates during regular operation when the treatment agent vaporizes and which is produced by cooling the steam generated by the high-speed steam generator at the vaporizer for the treatment agent is also fed to the feed vessel of the high-speed steam generator, so that a circuit for heating the vaporizer is created during regular operation.

Preferably, the treatment agent thus produced is used for sterilizing containers and/or container closures in the container treatment portion.

Further advantages and features of the present invention are apparent from the following description of various embodiments. The features described therein can be implemented on a stand-alone basis or in combination with one or more of the features set forth above, provided the features do not contradict one another. The following description of various embodiments is made with reference to the accompanying drawing.

### BRIEF DESCRIPTION OF THE DRAWING

Various embodiments of the invention will be explained in more detail by the following description of the FIGURE. In the drawing:

FIG. 1 schematically shows a system for treating containers with a container treatment portion and a high-speed steam generator.

### DETAILED DESCRIPTION

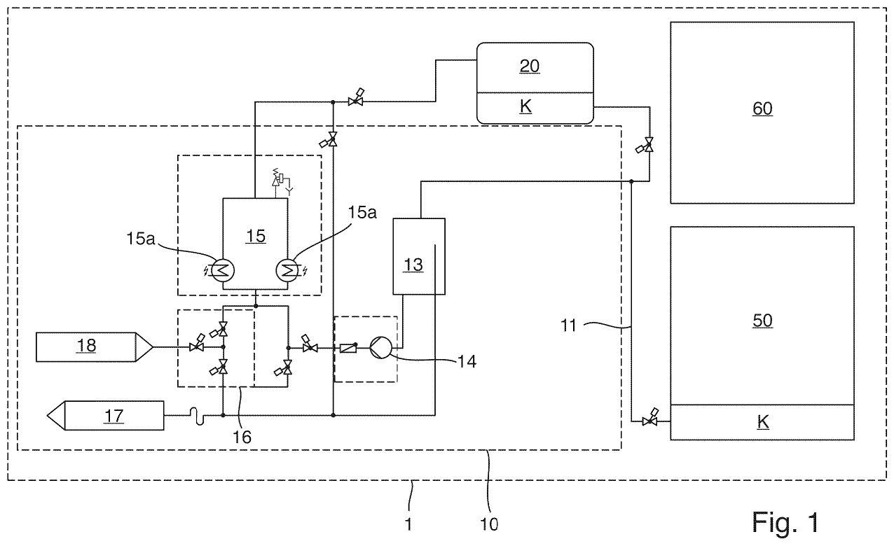

In the following, various embodiments are described with reference to FIG. 1, which schematically shows a system 1 for treating containers. The system 1 comprises a high-speed steam generator 10 and a container treatment portion 50.

The system 1 is particularly preferably a beverage filling system, the container treatment portion 50 in this case comprising in particular a filling device for filling water (still or carbonated), beer, juice, soft drinks, smoothies, dairy products, mixed drinks and the like. The container treatment portion 50 can comprise a filling device, a closing device, a stretch blow molding device for producing containers, a container cleaning system, a labeler, etc. or a combination of different devices, in particular a filling device for filling containers with a filling product and closing the containers with a container closure. The containers may be bottles made of glass, PET, cans, cartons or suitable containers of other types that can be filled with a liquid filling product, in particular a beverage.

The container treatment portion 50 can be supplied with operating steam from an operating steam generator 60, for example for a CIP (“Cleaning in Place”) treatment or SIP (“Sterilization in Place”) treatment, in which the container treatment portion 50 is cleaned and/or sterilized and/or disinfected on the product-contact surfaces without significant disassembly. The operating steam can perform alternative or additional functions in the container treatment portion 50, for example to heat certain parts of the system.

Typically, the operating steam is used at a comparatively low temperature, for example approx. 160° C., and a comparatively low pressure, for example approx. 6 bar.

The high-speed steam generator 10 can be conceived externally with respect to the container treatment portion 50 or can be partially or completely integrated into the container treatment portion 50, whereby it can be supplied with already existing media. Preferably, the high-speed steam generator 10 is integrated in terms of control technology into the container treatment portion 50, in particular the filling system.

Condensate K from processes in the container treatment portion 50 is used as feed water for the high-speed steam generator 10 and is preferably supplied from the steam network of the operating steam generator 60, i.e., in particular from the steam network with lower pressure. For this purpose, condensate K from the container treatment portion 50 is fed to the high-speed steam generator 10 via a condensate line 11.

The condensate K received via the condensate line 11 is fed to a feed vessel 13 in which it is caught and collected. This supply of condensate to the feed vessel 13 usually takes place while the condensate K is accumulating during sterilization in the container treatment portion 50.

In order to then remove the condensate from the feed vessel 13 during regular operation of the container treatment device, it is pumped from the feed vessel 13 into a steam boiler 15 via a pressure booster pump 14. The steam boiler 15 has heating elements 15a, preferably electrical heating elements, to heat the condensate K in the steam boiler 15. The heating elements 15a are regulated to the desired steam pressure. Furthermore, the steam boiler 15 preferably has safety devices to protect the steam boiler 15 in accordance with the specifications.

The steam thus generated, which preferably has a higher temperature and a higher pressure, for example 180° C. and 9 bar, than the steam produced by the operating steam generator 60, can be used in various processes. In particular, the steam can be used to operate, in particular to control the temperature, of a vaporizer 20, which is used, for example, to produce gaseous hydrogen peroxide for disinfecting lids or containers before they are actually filled with filling product.

The high-speed steam generator 10 can be connected to a cleaning circuit of the system 1 or the container treatment portion 50 via a CIP feed line 18, whereby the high-speed steam generator 10 can be integrated into an existing cleaning cycle in order to allow, for example, acid cleaning of the high-speed steam generator 10. The connection to the cleaning circuit is preferably made via a block & bleed valve combination 16 to prevent the media from mixing in the event of a valve leak.

The condensate K collected in the feed vessel 13 can, if necessary, be drained via a drain pipe 17 if emptying of the feed vessel is necessary.

During the above-mentioned CIP treatment of the container treatment portion 50, in particular using the operating steam generator 60, condensate K accumulates in the container treatment portion 50. The condensate K is then collected in the feed vessel 13 of the high-speed steam generator 10 until later use.

The condensate K is thus collected during system cleaning and used at a later time. For example, several hours may elapse between the collection of condensate K and the use of the condensate.

On this basis, the condensate K collected in the feed vessel 13 is used during the regular operation of the container treatment portion 50. In this case, the condensate K collected in the feed vessel 13 is fed to the steam boiler 15 during regular operation of the container treatment portion 50, wherein the steam generated in the steam boiler 15 is then used to heat the vaporizer for vaporizing the treatment agent, preferably hydrogen peroxide, and the vaporized treatment agent is fed to the container treatment portion 50 during regular operation.

The condensate K which accumulates during regular operation due to the cooling of the steam at the vaporizer is fed back into the feed vessel 13. In other words, a circulation of the condensate can be achieved during regular operation.

Preferably, the treatment agent thus produced is used for sterilizing containers and/or container closures in the container treatment portion 50.

The high-speed steam generator 10 is operated at least partially, preferably exclusively, with condensate K. This means that no separate water treatment is required or that any water treatment can be carried out with less effort, since the condensate K already has a very low mineral content.

The condensate K already has a high temperature, for example approx. 90° C., which saves energy and accelerates steam generation compared to cold feed water.

The possible connection to an existing cleaning cycle, preferably an acid cleaning cycle, leads to longer service life without the need for a corresponding blow-down process. Any mineral residues that may accumulate on the heating elements 15a can be automatically cleaned off during the next cleaning cycle of the container treatment portion 50.

To the extent applicable, any of the individual features set forth in the embodiments may be combined and/or interchanged without departing from the scope of the invention.

### LIST OF REFERENCE SIGNS

1 system for treating containers
10 high-speed steam generator
11 condensate line
13 feed vessel
14 pressure booster pump
15 steam boiler
15a heating element
16 block & bleed valve combination
17 drain pipe
18 CIP feed line
20 vaporizer
50 container treatment portion
60 operating steam generator
K condensate

1. A system (1) for treating containers, preferably a beverage filling system, which comprises:
a container treatment portion (50) which is configured to treat the containers, preferably to fill the containers with a filling product and/or to close the containers with a container closure, and in which condensate (K) accumulates during sterilization;
a high-speed steam generator (10) with a steam boiler (15) which is configured to heat feed water, preferably by means of at least one electrical heating element (15a), and to vaporize it, wherein
the high-speed steam generator (10) is configured to receive the condensate (K) accumulating in the container treatment portion (50) via a condensate line (11) and to introduce it into the steam boiler (15) as feed water to be vaporized during regular operation.

2. The system (1) according to claim 1, wherein the high-speed steam generator (10) has a feed vessel (13) which is configured to receive the condensate (K) via the condensate line (11), to collect it and to deliver it to the steam boiler (15), wherein the condensate (K) can preferably be conveyed from the feed vessel (13) into the steam boiler (15) by means of a pressure booster pump (14).

3. The system (1) according to claim 1, wherein the system (1) has a vaporizer (20) which is configured to receive steam from the steam boiler (15) of the high-speed steam generator (10) and to vaporize a treatment agent, preferably hydrogen peroxide, wherein the vaporizer (20) is preferably configured to introduce the vaporized treatment agent into the container treatment portion (50).

4. The system (1) according to claim 1, wherein the system (1) has an operating steam generator (60) which is different from the high-speed steam generator (10) and is configured to generate operating steam whose temperature and/or pressure are preferably lower than the temperature and/or pressure of the steam produced by the high-speed steam generator (10).

5. The system (1) according to claim 1, wherein the operating steam generator (60) is part of a cleaning circuit which is configured to clean and/or sterilize and/or disinfect the container treatment portion (50) as part of a CIP and/or SIP treatment, wherein condensate (K) accumulates during the CIP and/or SIP treatment.

6. The system (1) according to claim 1, wherein the high-speed steam generator (10) is connected to the cleaning circuit of the system (1) via a CIP feed line (18), wherein the connection to the cleaning circuit is preferably made via a block & bleed valve combination (16).

7. A method for providing steam in a system (1) for treating containers, preferably in a beverage filling system, comprising:
generating condensate (K) in a container treatment portion (50) during sterilization of the container treatment portion (50) which is configured to treat the containers, preferably to fill the containers with a filling product and/or to close the containers with a container closure;
during regular operation, introducing the condensate (K) via a condensate line (11) into a steam boiler (15) of a high-speed steam generator (10);
heating and evaporating the condensate (K) as feed water in the steam boiler (15), preferably by means of at least one electrical heating element (15a).

8. The method according to claim 7, wherein the high-speed steam generator (10) has a feed vessel (13) which receives the condensate (K) via the condensate line (11), collects it and delivers it to the steam boiler (15), wherein the condensate (K) is preferably conveyed from the feed vessel (13) into the steam boiler (15) by means of a pressure booster pump (14).

9. The method according to claim 7, wherein the steam generated in the steam boiler (15) drives a vaporizer (20) which vaporizes a treatment agent, preferably hydrogen peroxide, wherein the vaporizer (20) provides the vaporized treatment agent for the treatment of containers to be filled and/or container closures.

10. The method according to claim 7, wherein an operating steam generator (60) which is different from the high-speed steam generator (10) produces operating steam whose temperature and/or pressure are preferably lower than the temperature and/or pressure of the steam produced by the high-speed steam generator (10).

11. The method according to claim 10, wherein the operating steam generator (60) is part of a cleaning circuit which cleans and/or sterilizes and/or disinfects the container treatment portion (50) as part of a CIP and/or SIP treatment.

12. The method according to claim 8, wherein the condensate (K) accumulates during the CIP and/or SIP treatment of the container treatment portion (50), wherein the condensate (K) is collected in the feed vessel (13) of the high-speed steam generator (10) until later use.

13. The method according to claim 8, wherein the condensate (K) collected in the feed vessel (13) is fed to the steam boiler (15) during regular operation of the container treatment portion (50), wherein the steam thus generated in the steam boiler (15) is used in the vaporizer (20) to vaporize the treatment agent, preferably hydrogen peroxide, and the vaporized treatment agent is fed to the container treatment portion (50) during regular operation, preferably for sterilizing containers and/or container closures.
